(19) United States
(12) Patent Application Publication
(10) Pub. No.: US 2019/0262577 A1
(43) Pub. Date: Aug. 29, 2019

(54) PACKAGING FOR MEDICAL DEVICE

(71) Applicant: BOSTON SCIENTIFIC SCIMED, INC., Maple Grove, MN (US)

(72) Inventor: JOEL RICHARD ANDERSON, MAPLE GROVE, MN (US)

(73) Assignee: BOSTON SCIENTIFIC SCIMED, INC., MAPLE GROVE, MN (US)

(21) Appl. No.: 16/289,245

(22) Filed: Feb. 28, 2019

Related U.S. Application Data
(60) Provisional application No. 62/636,503, filed on Feb. 28, 2018.

(51) Int. Cl. A61M 25/00; A61B 50/30

(52) U.S. Cl. CPC A61M 25/002; A61B 50/20; A61L 2202/24; A61B 50/30

(57) ABSTRACT

Devices, assemblies, systems and methods for packaging an elongated medical device including a securement member for securing the medical device within the packaging. The packaging and/or assembly may include a carrier tube configured to receive a portion of the elongated medical device within a lumen thereof, and a securement member coupled to the carrier tube for securing the medical device relative to the carrier tube. The securement member may include a body defining a tapered channel extending into the body, the tapered channel including an inner surface configured to engage an outer surface of the medical device.

### CROSS-REFERENCE TO RELATED APPLICATIONS

This application claims the benefit of priority under 35 U.S.C. § 119 of U.S. Provisional Application No. 62/636,503, filed Feb. 28, 2018, the entire disclosure of which is hereby incorporated by reference.

### TECHNICAL FIELD

The present disclosure pertains to medical devices, medical device packaging, and methods for preparing and packaging medical devices. More particularly, the present disclosure pertains to packaging for elongated intracorporeal medical devices including a securement member for securing a medical device relative to a carrier tube, and methods for manufacturing and using such packaging.

### BACKGROUND

A wide variety of intracorporeal medical devices have been developed for medical use, for example, intravascular use. Some of these devices include guidewires, catheters, and the like. These devices are manufactured and packaged using any one of a variety of different packaging assemblies and/or methods. Of known packaging and methods of packaging medical devices, each may have certain advantages and disadvantages. There is an ongoing need to provide alternative medical device packaging, as well as alternative methods for packaging and/or preparing medical devices.

### BRIEF SUMMARY

This disclosure provides packaging design, material, manufacturing, and/or methods for use in packaging medical devices. An example includes a packaging assembly for an elongated medical device. The assembly includes a carrier tube having a first end with a first opening, a second end with a second opening, and a tube body defining a lumen extending between the first opening and the second opening, the carrier tube configured to receive a portion of the elongated medical device within the lumen. The assembly includes a securement member coupled to the carrier tube, the securement member including a body defining a tapered channel extending into the body, the tapered channel including an inner surface configured to engage an outer surface of the medical device.

Alternatively or additionally to any of the embodiments above or below, wherein the inner surface of the tapered channel is configured to releasably engage the outer surface of the medical device such the medical device is releasably secured to the securement device.

Alternatively or additionally to any of the embodiments above or below, wherein the inner surface of the tapered channel is configured to form an interference fit with the outer surface of the medical device.

Alternatively or additionally to any of the embodiments above or below, wherein the tapered channel of the securement member has a first end and a second end, and the diameter of the tapered channel is larger at the first end than at the second end.

Alternatively or additionally to any of the embodiments above or below, wherein the tapered channel of the securement member has an opening defined by the body of the securement member, and the securement member is coupled to the carrier tube such that the opening of the securement member is aligned with the first opening of the carrier tube.

Alternatively or additionally to any of the embodiments above or below, wherein the tapered channel of the securement member has a first end including an opening defined by the body of the securement member, and a second closed end.

Alternatively or additionally to any of the embodiments above or below, wherein the tapered channel gradually tapers in diameter from a first end to a second end.

Alternatively or additionally to any of the embodiments above or below, wherein the securement member coupled to the carrier tube adjacent the first opening in the carrier tube.

Alternatively or additionally to any of the embodiments above or below, wherein the securement member is coupled to the carrier tube by a clip.

Another example is a packaged medical device, including a carrier tube having a first end with a first opening, a second end with a second opening, and a tube body defining a lumen extending between the first opening and the second opening; a securement member coupled to the carrier tube, the securement member including a body defining a tapered channel extending into the body, the tapered channel including an inner surface; and an elongated medical device including a body portion disposed within the lumen of the carrier tube and including an end portion disposed within the securement member, the end portion having an outer surface that engages the inner surface of the tapered channel.

Alternatively or additionally to any of the embodiments above or below, wherein the inner surface of the taped channel is releasably engaged with the outer surface of the medical device such the medical device is releasably secured to the securement device.

Alternatively or additionally to any of the embodiments above or below, wherein the inner surface of the tapered channel is frictionally engaged with the outer surface of the medical device.

Alternatively or additionally to any of the embodiments above or below, wherein the securement member is coupled to the carrier tube adjacent the first opening in the carrier tube, and the medical device extends out of the opening in the carrier tube and into the channel of the securement member.

Another example is a method of packaging an elongated medical device including a body portion and an end portion. The example method includes disposing the body portion of the elongate medical device in a lumen of a carrier tube, the carrier tube having a first end with a first opening, a second end with a second opening, and a tube body defining the lumen extending between the first opening and the second opening; and disposing the end portion of the medical device into a tapered channel of a securement member such that an outer surface of the end portion of the medical device engages an inner surface of the tapered channel, the securement member being coupled to the carrier tube and including a body defining the tapered channel.

Alternatively or additionally to any of the embodiments above or below, wherein the inner surface of the tapered channel is configured to frictionally engage the outer surface of the medical device such the medical device is releasably secured to the securement device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is schematic perspective view of a medical device disposed in a packaging assembly including a carrier tube and a securement member.

FIG. 2 is schematic perspective view of the securement member from FIG. 1.

FIG. 3 is a cross sectional view of the securement member showing a tapered channel, and an end of an elongated medical device outside of the cannel.

FIG. 4 is a cross sectional view of the securement member showing the tapered channel, and the end of an elongated medical device disposed within the cannel such that the inner surface of the channel engages the outer surface of the medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Elongated medical devices may be contained within appropriate packaging that allows the medical devices to be protected during shipping, storage, preparation, and/or usage. For example, prior to sale, elongated medical devices such as guidewires, catheters, and the like, may be sterilized and packaged. When suitably packaged, the elongated medical devices can be transported to an appropriate medical setting where they can be stored until needed for use. Because devices may be susceptible to damage when shipped and/or stored, some or all of the device may be disposed within a tube or shell often termed a carrier tube. The carrier tube may be part of the packaging that affords the device some level of protection during the shipping/storage phase as well as during preparation or use prior to or during a clinical intervention. Other desirable benefits may also be achieved through the use of a carrier tube as part of the packaging. For example, the carrier tube may be coiled, and maintain the elongated medical device in a configuration that may be easier and/or more convenient to store. Further, a carrier tube may help protect the elongated device during removal of the device from other packaging (e.g. box or pouch, etc.). Additionally, the use of a carrier tube may aid a user to better maintain the sterility and control of the device during an operation.

It may be desirable that the elongate medical device be selectively and/or releasably secured within the carrier tube (e.g. secured from relative proximal and/or distal movement within the career tube), but also allow for the selective removal of the elongated medical device from the carrier tube by a user, when desired. Thus a securement member and/or mechanism is desirable that provides for selective and/or releasable securement of the elongated medical device relative to the carrier tube. It may also be desirable that the securement member and/or mechanism does not expose the medical device to substantial risk of damage during selective engagement/disengagement of the medical device with the securement member and/or mechanism. Furthermore, it may be desirable to provide a securement member and/or mechanism that may protect and/or house an end of the elongated medical device. Disclosed herein are a number of medical device packaging, packaging assemblies and/or packaging components, and methods for packaging and/or preparing a medical device. In at least some embodiments, the packages, packaging components, and methods disclosed herein may allow for or otherwise include a securement member and/or mechanism. Some of the example securement members and/or mechanisms may include one or more of these features and/or achieve one or more of the advantages discussed above. Other features and advantages may also be appreciated, as disclosed herein.

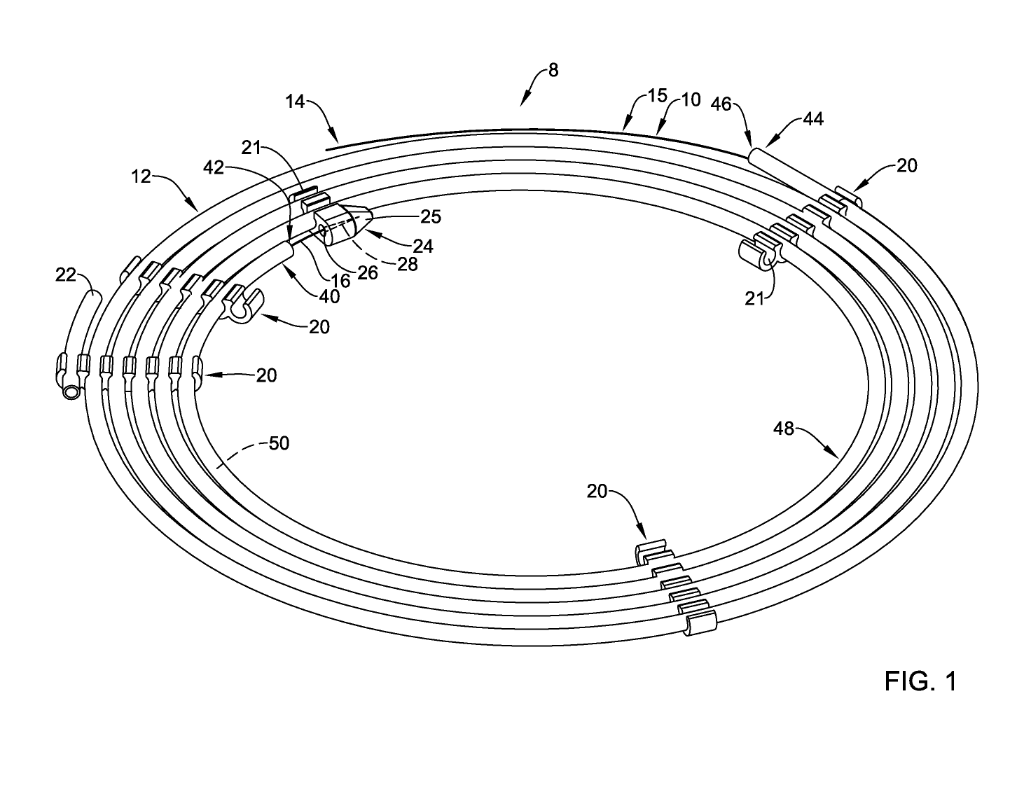

FIG. 1 illustrates an example embodiment, showing a perspective view of a medical device 10 disposed in a packaging assembly 8 including a carrier tube 12 and a securement member 24. In this example, the medical device 10 may be a guidewire. In other embodiments, medical device 10 may be another type of elongated medical device, such as a catheter or the like. The elongated medical device 10 includes a first end portion 16 and a second end portion 14, and a body portion 15 disposed between the two end portions. In FIG. 1, a large part of the body portion 15 is disposed within, and thereby covered by, the carrier tube 12. In some embodiments, only a portion of the body 15 is disposed within the carrier tube 12, while in other embodiments the entire body portion 15 may be disposed within the carrier tube 12. In some embodiments, the first end portion 16 may be a proximal end portion and may include the proximal most terminus of the medical device 10, and the second end portion 14 may be a distal end portion and may include the distal-most terminus of the medical device 10. In other embodiments, this may be reversed (e.g. the first end portion may be the proximal portion of the medical device and the second end portion may be the distal portion).

The carrier tube 12 includes a first end 40 with a first opening 42, a second end 44 with a second opening 46, and a tube body 48 defining a lumen 50 extending between the first opening 42 and the second opening 46. The carrier tube 12 is configured to receive a portion of the elongated medical device 10 within the lumen 50. For example, as shown in FIG. 1, the body portion 15 is disposed within the lumen 50 of the carrier tube 12.

The carrier tube 12 may generally be configured to hold the medical device 10 in a suitable configuration. In at least some embodiments, the carrier tube 12 may be arranged or otherwise configured as a coil that allows the medical device 10, which may have a reasonably long length, to be held in a compact configuration. The individual windings of the coil may be secured together by one or more clips 20. For example, each of the clips 20 may include two or more clip portions 21, each clip portion 21 configured to selectively and/or releasably engage or grip a portion of the carrier tube 12 to hold it in the desired configuration (e.g. coiled). Optionally, a carrier tube stub portion 22, which may simply be a shortened section of tube connected to one of the outer clips 20, may also be included. Such a portion 22 in combination with the carrier tube 12, may be used, for example, in maintaining control of an end portion of the medical device, while allowing a section of the medical device to be exposed for manipulation by a user. Other configurations for the carrier tube 12 include configurations suitable for holding other medical devices, as desired.

The packaging assembly includes a securement member 24 coupled and/or attached to the carrier tube 12 for selectively and/or releasably securing the medical device 10 within and/or relative to the carrier tube 12. The securement member 24 may include a body portion 25. The body portion 25 may define an opening 26 that provides a pathway into a tapered cavity and/or channel 28 defined by the body portion 25. The channel 28 may be configured to receive an end portion (e.g. 16) of the medical device 10, and secure the end portion of the medical device 10 therein—thereby securing the medical device 10 relative to the carrier tube 12. (as will be discussed in more detail below).

The securement member 24 may be coupled to the carrier tube 12 such that it is oriented in a desired configuration and/or orientation. In particular, the securement member 24 may be coupled to the carrier tube 12 such that the opening 26 and/or the tapered channel 28 in the securement member 24 is adjacent to and/or aligned with an opening 42 in the carrier tube 12. For example, as shown in FIG. 1, the opening 26 in the securement member 24 is adjacent to and/or aligned with the first opening 42 of the carrier tube 12. The two openings 42 and 26 may be aligned such that they face each other. The openings 26 and 42 may be aligned such that a portion of the medical device 10 (e.g. first end portion 16) extending out of the opening 42 of the carrier tube 12 may be aligned with and extend into the opening 26 of the securement member 24, and into the tapered cavity and/or channel 28. In some embodiments, the carrier tube 12 may extend about and/or along a first longitudinal axis (e.g. about and/or along the lumen 50 and/or first opening 42 of the carrier tube 12), and securement member 24 may extend along and/or about a second longitudinal axis (e.g. about and/or along the opening 26 and/or the tapered channel 28 of the securement member 24), and the securement member 24 may be coupled to the carrier tube 12 such that these two longitudinal axes are in alignment. It is also contemplated in other embodiments that the securement member 24 may be coupled to the carrier tube 12 such the opening 26 of the securement member 24 may be aligned with the second opening 46 of the carrier tube 12, such that a portion of the medical device 10 (e.g. second end portion 14) extending out of the opening 46 may be aligned with and extend into the opening 26 of the securement member 24, and into the tapered cavity and/or channel 28.

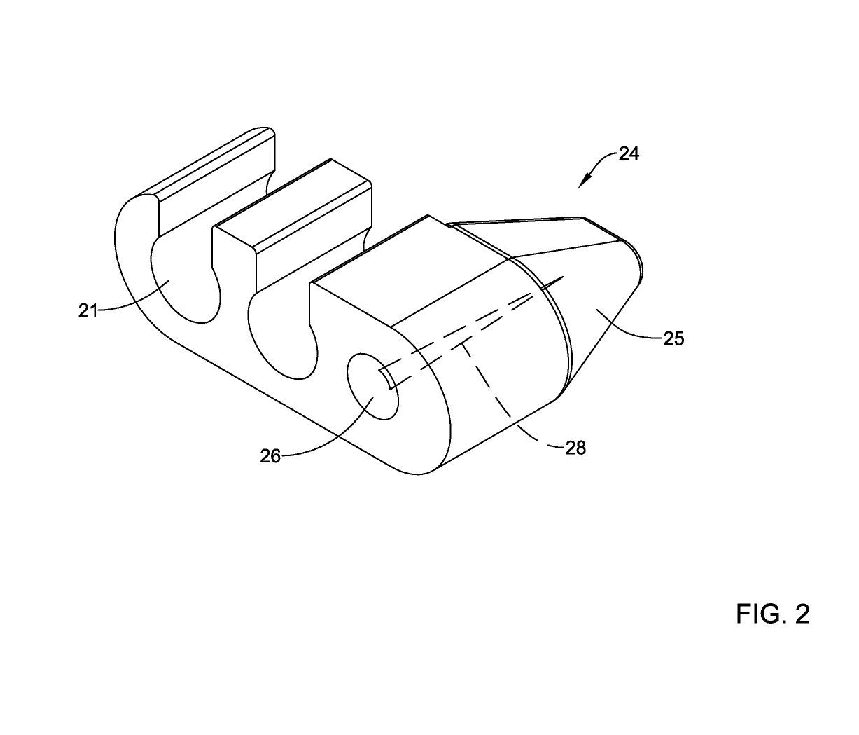

The securement member 24 may be coupled to the carrier tube 12 using any mechanism and/or structure as desired. In the embodiment shown, for example in FIGS. 1 and 2, the body portion 25 includes one or more clip portions 21, each clip portion 21 configured to selectively and/or releasably engage or grip a portion of the carrier tube 12, to thereby couple the securement member 24 to the carrier tube 12. The clip portions 21 may be of monolithic and/or integral construction with the body 25 of the securement member 24. Other configurations for coupling and/or attaching the securement member 24 and the carrier tube 12 are contemplated. For example, it is contemplated that securement member 24 may be part of and/or permanently attached to carrier tube 12 in the desired orientation, rather than releasably attached as discussed above. Alternatively, it is contemplated that the carrier tube 12 may include integral and/or permanent structure (e.g. clips) configured to releasably engage and/or hold a separate securement member 24 in the correct orientation relative thereto. In yet another contemplated alternative, a separate coupling member and/or mechanism that includes one or more separate parts may be used to interconnect the carrier tube 12 and the securement member 24 in the desired orientation.

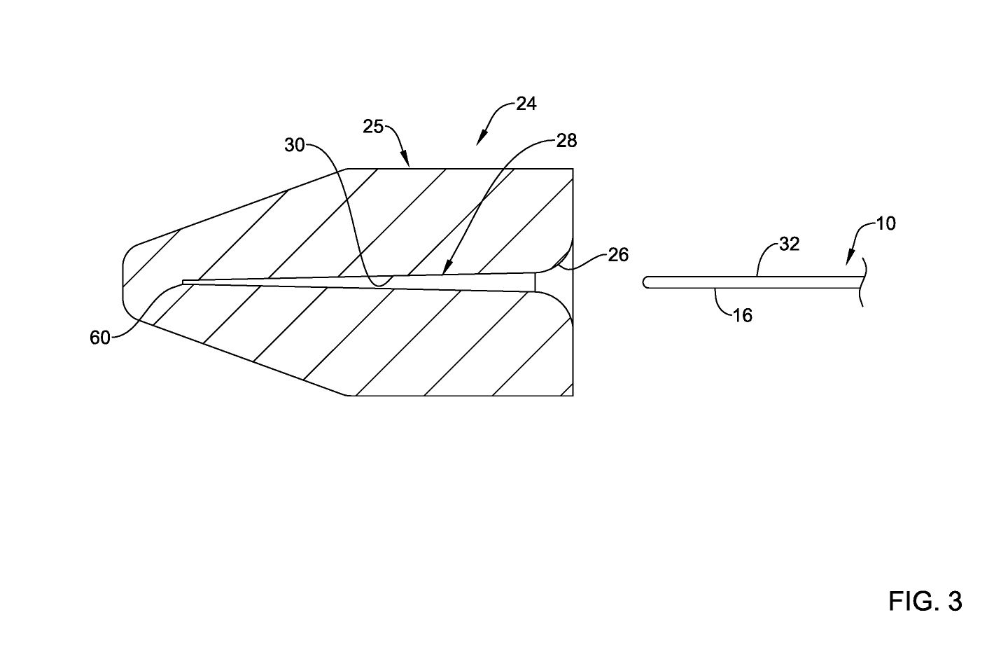

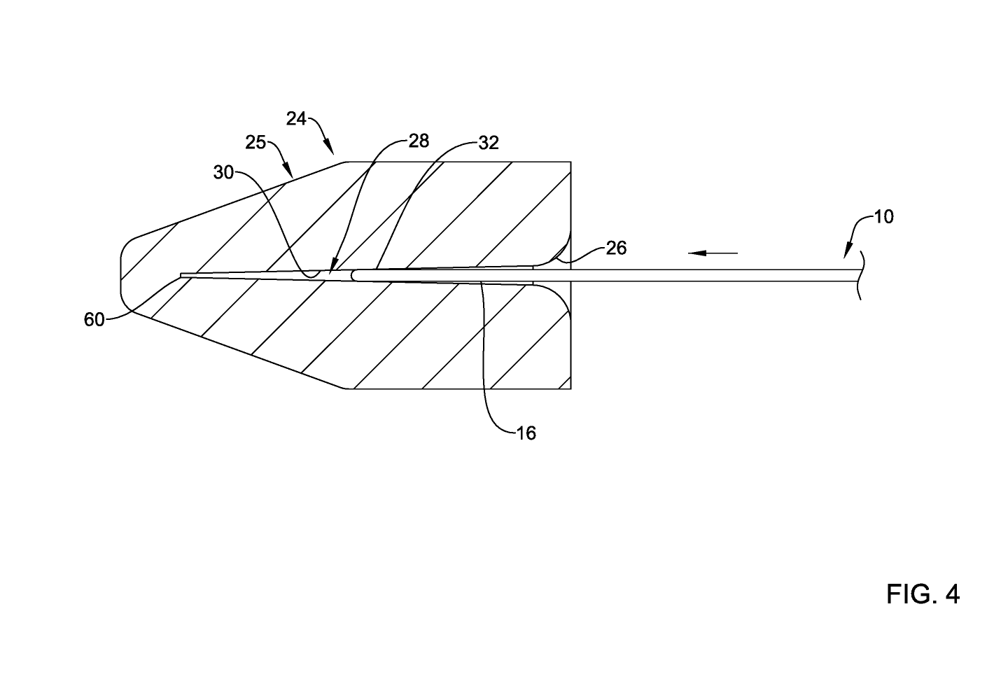

As shown in cross sectional view of FIGS. 3 and 4, the tapered cavity and/or channel 28 is defined by and extends into the body portion 25 from a first end at the opening 26 to a second end 60. In the embodiment shown, the second end 60 is a closed end. However, in other embodiments, the end 60 may be an open end, defining a second opening opposite the opening 26. The diameter of the tapered channel 28 changes and/or varies and/or tapers in along its length from the opening 26 to the end 60. In the embodiment shown, the diameter of the channel 28 is largest adjacent the opening 26, and tapers such that the dimeter is smallest adjacent the end 60.

The tapered channel 28 includes an inner surface 30 that is configured to engage an outer surface 32 of a portion of the medical device 10. For example, at some point along its length, the tapered cannel 28 includes a dimeter that is equal to and/or smaller than a diameter of a portion (e.g. a diameter of the end portion 16) of the medical device 10. As such, the when the portion (e.g. end portion 16) of the medical device is inserted into the opening 26 and advanced within the tapered cannel 28, at some point along its length, the inner surface 30 is configured to engage an outer surface 32 of the end portion 16 of the medical device 10. FIG. 3 shows the end portion 16 of the medical device 10 adjacent to the securement member 24 prior to insertion into the tapered channel 28. FIG. 4 then shows the end portion 16 of the medical device 10 inserted into the tapered channel 28 of the securement member 24, and advanced in the direction of the arrow until the inner surface 30 engages the outer surface 32. Due to the reducing diameter of the channel 28, at a point along its length, the inner surface 30 thereof engages the outer surface 32 of the end portion 16 of the medical device 10. This engagement can provide a securement force that selectively and/or releasably secures the longitudinal position of the medical device 10 within the securement member 24. Because the securement member 24 is coupled to the carrier tube 12, the longitudinal position of the medical device 10 is thereby selectively and/or releasably secured relative to the carrier tube 12 by the securement member 24. When it is desired to selectively release the medical device 10 from the securement member 24, a user simply pulls the medical device 10 in the opposite direction to disengage the outer surface 32 from the inner surface 30. The medical device 10 may them be removed from the securement member 24, and moved longitudinally relative to the carrier tube 12, for example, to remove it from the carrier tube 12 for use.

In some respects, the engagement between the inner surface 30 and the outer surface 32 may be characterized as a mechanical interlock and/or interference fit and/or pinch fit. Due to the progressive reduction of the tapered channel 28 to an inner diameter that is less than the outer diameter of the end portion 16 of the medical device 10, advancement of the end portion 16 therein will at some point put the two surfaces (inner 30 and outer 32) into engagement. After this initial point of engagement, some additional amount of force applied in the advancement/engagement direction will create a selectively releasable interference fit between the two structures. In essence, forcing a larger diameter end portion 16 of the medical device 10 into the progressively smaller diameter passage (e.g. the channel 28) with a predetermined amount of force will create such a selectively releasable interference fit between the medical device 10 and the securement member 24.

The amount of insertion force used to create the securement between the medical device 10 and the securement member 24 should be at a sufficient level to create the desired level of engagement and/or securement between the medical device 10 and the securement member 24, but not at a level sufficient to damage the medical device 10. Similarly, the level engagement between the surfaces and/or the level of force needed to disengage the medical device 10 from the securement member 24 should not be high enough to possibly damage the medical device 10. As such, it is desired to use a sufficient amount of engagement/disengagement force to create the desired level of releasable securement, but not too much—where damage to the medical device 10 may be possible.

In some embodiments, the angle of taper of the tapered channel 28 can be configured to enhance this interference and/or pinch fit. Some example angles of the taper along the inner surface 30, relative to the longitudinal axis of the channel 28, may include in the range of 1 to 10 degrees, or in the range of 2 to 8 degrees, or in the range of 3 to 6 degrees. The tapered channel 28 may include a constant taper angle along its length, or may include two or more tapered angles separated by constant diameter sections, or a compound taper angle, or a series of taper angles along its length, and may be tapered in a stepwise manner, or in progressively and/or constantly changing manner. In some embodiments, the depth of the tapered channel 28 may also be configured to enhance the interference and/or pinch fit. Some example depth measurements for the tapered channel 28 may include in the range of 0.1 to 1 inch, or in the range of 0.2 to 0.8 inch, or in the range of 0.2 to 0.6 inch.

In some embodiments, the inner surface 30 of the tapered channel 28 may include or be made of a material that may enhance the engagement with the outer surface 32 of the medical device. For example, the inner surface 30 may be made of or include a more deformable and/or softer material. (e.g. lower durometer material). Such softer and/or more deformable material may provide for a better “grab” of the medical device 10 during engagement/securement. Some example materials may include polymer materials having a durometer in the range of 25 to 90, or in the range of 30 to 80.

In some respects, the engagement between the inner surface 30 and the outer surface 32 may alternatively and/or additionally be characterized as a friction fit. In other words, frictional engagement of the inner surface 30 and the outer surface 32 (in addition to and/or in combination with an interference fit and/or pinch fit) may provide for the desired releasable securement of the medical device 10 to the securement member 24. In this context, the two surfaces 30 and 32 may be in frictional engagement with one another. In some embodiments, the surfaces 30 and 32 may include surface features and/or texturing to provide for and/or enhance the frictional engagement therebetween.

As may also be appreciated, for example as shown in FIG. 4, the securement member 24 may also house the end portion 16 of the medical device 10 when disposed therein. This may aid in preventing damage to the end portion and/or tip.

As can also be appreciated, the packaging assembly 8, including the medical device 10, the carrier tube 12, and/or the securement member 24, may include additional packaging components. For example, the packaging assembly 8 may be disposed in a pouch, box, barrier, etc. . . . . The housing of the end portion 16 (including the tip) of the medical device 10 within the securement member 24 may also help prevent the end portion 16 from causing puncturing and/or causing damage to such other packaging material.

The securement member 24 may be formed in a suitable manner. For example, the securement member 12 may be formed via molding, casting, or other suitable process. In some embodiments, the opening 26, tapered channel 28, and/or clip portions 21 may be formed in the securement member 24 during the forming of the securement member, for example during molding. In other embodiments, the body 25 of the securement member may be formed first, and the opening 26, tapered channel 28, and/or clip portions 21 may be formed in body 25 of the securement member 24 by machining, cutting, drilling, laser cutting, etching, scoring, or other suitable manners.

Similarly, the clips 20 may be formed in a suitable manner. For example, the clips 20 may be formed via molding, casting, or other suitable process. In some embodiments, the clip portions 21 of the clips 20 may be formed during the formation of the clips 20, for example during molding. In other embodiments, the body of the clips 20 may be formed first, and the clip portions 21 may be formed by machining, cutting, drilling, laser cutting, etching, scoring, or other suitable manners.

Carrier tube 12 may be formed in a suitable manner. For example, carrier tube 12 may be formed via an extrusion process. Alternatively, carrier tube 12 may be formed via molding, casting, or other suitable process.

The securement member 24, the carrier tube 12 and/or the clips 20 may generally be formed of any material as desired, but in some embodiments, may be formed of a polymer material. Some examples of suitable polymers include thermoplastic vulcanizate (TPV), such as Santoprene™; polytetrafluoroethylene (PTFE); ethylene tetrafluoroethylene (ETFE); fluorinated ethylene propylene (FEP); polyoxymethylene (POM), for example, DELRIN® available from DuPont); polyether block ester, polyurethane; polypropylene (PP); polyvinylchloride (PVC); polyether-ester (for example, ARNITEL® available from DSM Engineering Plastics); ether or ester based copolymers (for example, butylene/poly(alkylene ether) phthalate and/or other polyester elastomers such as HYTREL® available from DuPont); polyamide (for example, DURETHAN® available from Bayer or CRISTAMID® available from Elf Atochem); elastomeric polyamides; block polyamide/ethers; polyether block amide (PEBA, for example available under the trade name PEBAX®); ethylene vinyl acetate copolymers (EVA); silicones; polyethylene (PE); high-density polyethylene (for example MARLEX® high-density polyethylene); low-density polyethylene (for example MARLEX® low-density polyethylene); linear low density polyethylene (for example REXELL®); polyester; polybutylene terephthalate (PBT); polyethylene terephthalate (PET); polytrimethylene terephthalate; polyethylene naphthalate (PEN); polyetheretherketone (PEEK); polyimide (PI); polyetherimide (PEI); polyphenylene sulfide (PPS); polyphenylene oxide (PPO); poly paraphenylene terephthalamide (for example, KEVLAR®); polysulfone; nylon; nylon-12 (such as GRILAMID® available from EMS American Grilon); perfluoro(propyl vinyl ether) (PFA); ethylene vinyl alcohol; polyolefin; polystyrene; epoxy, polyvinylidene chloride (PVdC); polycarbonates; ionomers; biocompatible polymers, other suitable materials, or mixtures, combinations, copolymers thereof, polymer/metal composites, and the like. In some embodiments the sheath can be blended with a liquid crystal polymer (LCP).

The securement member 24, the carrier tube 12 and/or the clips 20 may also be made or and/or include other materials such as metals, metal alloys, metal-polymer composites, or the like. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A packaging assembly for an elongated medical device, the assembly comprising:
a carrier tube having a first end with a first opening, a second end with a second opening, and a tube body defining a lumen extending between the first opening and the second opening, the carrier tube configured to receive a portion of the elongated medical device within the lumen; and
a securement member coupled to the carrier tube, the securement member including a body defining a tapered channel extending into the body, the tapered channel including an inner surface configured to engage an outer surface of the medical device.

2. The assembly of claim 1, wherein the inner surface of the tapered channel is configured to releasably engage the outer surface of the medical device such the medical device is releasably secured to the securement device.

3. The assembly of claim 1, wherein the inner surface of the tapered channel is configured to frictionally engage the outer surface of the medical device.

4. The assembly of claim 1, wherein the tapered channel of the securement member has a first end and a second end, and the diameter of the tapered channel is larger at the first end than at the second end.

5. The assembly of claim 1, wherein the tapered channel of the securement member has an opening defined by the body of the securement member, and the securement member is coupled to the carrier tube such that the opening of the securement member is aligned with the first opening of the carrier tube.

6. The assembly of claim 1, wherein the tapered channel of the securement member has a first end including an opening defined by the body of the securement member, and a second closed end.

7. The assembly of claim 1, wherein the tapered channel continuously tapers in diameter from a first end to a second end.

8. The assembly of claim 1, wherein the securement member is coupled to the carrier tube adjacent the first opening in the carrier tube.

9. The assembly of claim 1, wherein the securement member is releasably coupled to the carrier tube.

10. The assembly of any of claim 1, wherein the securement member is coupled to the carrier tube by a clip.

11. The assembly of claim 10, wherein the clip is of monolithic construction with the securement member.

12. The assembly of claim 10, wherein the inner surface of the tapered channel includes a surface texture configured to releasably engage the outer surface of the medical device.

13. The assembly of claim 10, wherein the inner surface of the tapered channel is made of a material having a durometer configured to releasably engage the outer surface of the medical device.

14. The assembly of claim 10, wherein the tapered channel extends along a longitudinal axis, and the inner surface of the tapered channel is tapered at a taper angle relative to the longitudinal axis, and the taper angle is configured to allow the inner surface of the tapered channel releasably engage the outer surface of the medical device.

15. A packaged medical device comprising:
a carrier tube having a first end with a first opening, a second end with a second opening, and a tube body defining a lumen extending between the first opening and the second opening;
a securement member coupled to the carrier tube, the securement member including a body defining a tapered channel extending into the body, the tapered channel including an inner surface; and
an elongated medical device including a body portion disposed within the lumen of the carrier tube and including an end portion disposed within the securement member, the end portion having an outer surface that engages the inner surface of the tapered channel.

16. The packaged medical device of claim 10, wherein the inner surface of the taped channel is releasably engaged with the outer surface of the medical device such the medical device is releasably secured to the securement device.

17. The packaged medical device of any of claims 10 or 11, wherein the inner surface of the tapered channel is frictionally engaged with the outer surface of the medical device.

18. The packaged medical device of any of claims 10-12, wherein the securement member is coupled to the carrier tube adjacent the first opening in the carrier tube, and the medical device extends out of the opening in the carrier tube and into the channel of the securement member.

19. A method of packaging an elongated medical device including a body portion and an end portion, the method comprising:
disposing the body portion of the elongate medical device in a lumen of a carrier tube, the carrier tube having a first end with a first opening, a second end with a second opening, and a tube body defining the lumen extending between the first opening and the second opening; and
disposing the end portion of the medical device into a tapered channel of a securement member such that an outer surface of the end portion of the medical device engages an inner surface of the tapered channel, the securement member being coupled to the carrier tube and including a body defining the tapered channel.

20. The method of claim 34, wherein the inner surface of the tapered channel is configured to frictionally engage the outer surface of the medical device such the medical device is releasably secured to the securement device.
